(19) United States
(12) Patent Application Publication
(10) Pub. No.: US 2023/0106928 A1
(43) Pub. Date: Apr. 6, 2023

(54) Undulating Balloon Systems and Methods for Nanoparticle-Based Drug Delivery

(71) Applicant: Advanced NanoTherapies, Inc., Cleveland, OH (US)

(72) Inventors: Marwan Berrada-Sounni, Los Gatos, CA (US); Sean T. Zuckerman, Lakewood, OH (US)

(21) Appl. No.: 17/908,479

(22) PCT Filed: Mar. 2, 2021
(86) PCT No.: PCT/US2021/020545

§ 371 Date: Aug. 31, 2022

Related U.S. Application Data
(60) Provisional application No. 62/983,921, filed on Mar. 2, 2020.

(51) Int. Cl. A61B 17/22

(52) U.S. Cl. CPC A61B 17/22; A61B 2017/22001; A61B 2017/22014; A61B 2017/22051

(57) ABSTRACT

Systems and methods for localized drug delivery via undulating drug coated balloons (DCB), in particular using functionalized nanoparticles as a drug delivery medium in combination with an undulating balloon, are disclosed. In various disclosed embodiments, a nanoparticle matrix is adhered to in an external substrate-surface, such as the balloon surface, and is activated for release once at the treatment site. Activation for release may be enhanced through the use of an undulating balloon system including methodologies for precise control of timing, waveform and extent of undulations.

### RELATED APPLICATION DATA

This application claims the benefit of priority of U.S. Provisional Pat. Application Serial No. 62/983,921, filed Mar. 2, 2020, and titled Nanoparticle-Based Drug Delivery Therapeutic Devices and Methods, which is incorporated by reference herein in its entirety.

### FIELD OF THE DISCLOSURE

The present disclosure generally relates to the fields of drug delivery and drug coated balloons. In particular, the present disclosure is directed to undulating balloon systems and methods for nanoparticle-based drug delivery.

### BACKGROUND

Drug coated balloons (DCB) have been used for treatment of coronary artery disease and peripheral artery disease (PAD) for many years. In a conventional DCB, the drug payload is coated on the balloon using a wide variety of coating techniques. In use, similar to a plain old balloon angioplasty (POBA) procedure, the DCB is placed across the lesion and expanded to compress, and force drugs into, the lesion. While some success has been achieved to date with DCBs, one limitation is challenges in drug delivery from the balloon to the arterial wall and adequate retention of the initially delivered drug for a time sufficient to have a lasting beneficial effect.

A number of different devices or techniques have been proposed in an attempt to improve results of angioplasty procedures. In one proposal, the PTA balloon is configured to vibrate at a relatively high frequency with a goal of fracturing or breaking up plaque forming the lesion. However, a drawback of vibrating balloons is believed to be stimulation of intimal thickening and proliferation of smooth muscle cells in the vessel wall as a result of the forceful, high frequency vibrations applied to break up the plaque of the lesion. Smooth muscle cell proliferation is undesirable because it can be a significant cause of stenosis or narrowing. Such high frequency vibrations may also impede rather than promote drug uptake via drug carrying media such as nanoparticles if implemented in a DCB.

Another limitation with conventional DCBs for treatment of PAD is limitation of the available drugs. For example, while both paclitaxel and sirolimus are known to show efficacy in limiting restenosis in PAD, to date sirolimus has not been generally accepted for use with DCBs because it has a much slower uptake by cells as compared to paclitaxel-rendering delivery via DCB far more challenging.

However, functionalized nanoparticles have shown significant promise as vehicles for delivery of a wide variety of drug compounds, including sirolimus. Examples of such nanoparticles are disclosed in US Pat. No. 8,865,216 to Labhasetwar et al., granted Oct. 21, 2014, and entitled “Surface-Modified Nanoparticles for Intracellular Delivery of Therapeutic Agents and Composition for Making Same, which is incorporated by reference in its entirety herein. There is, however, a need for effective delivery devices for drug carrying nanoparticles, particularly as regards to delivery of sirolimus and other drugs intravascularly, alone or in combinations for multiple drug treatments.

### SUMMARY OF THE DISCLOSURE

In one implementation, the present disclosure is directed to an undulating balloon PTA system. The system includes a balloon catheter having preset maximum inflation pressure, an oscillating fluid pressure source communicating with the balloon catheter, and a controller configured to cause the oscillating fluid pressure source to deliver controlled pressure oscillations to the balloon catheter between a maximum pressure equal to the preset maximum inflation pressure and a set minimum pressure at a selected cycle time. In some embodiments the set minimum pressure is not more than 50% less than the maximum pressure. In other embodiments, the controller is further configured to deliver the pressure oscillations at a cycle time of 10 seconds to about 0.25 seconds. In still other embodiments, the set minimum pressure is not more than 30% less than the maximum pressure and the controller is configured to deliver pressure oscillations at a cycle time of 1 second to about 0.25 seconds. In a further embodiment, the controller is configured to deliver pressure oscillations with a minimum cycle time setting of 0.5 seconds.

In another implementation, the present disclosure is directed to an undulating balloon PTA system comprising an inflatable balloon member with a drug-carrying nanoparticle matrix disposed on an outer surface of the balloon member. The drug-carrying nanoparticle matrix preferably contains microchannels, whereby blood may circulate in the microchannels to increase hydration of the nanoparticle matrix. In another embodiment, the nanoparticle matrix comprises drug-carrying nanoparticles and interstitial bonding agent, wherein the interstitial bonding agent is configured to release the drug-carrying nanoparticles in response to predetermined stimulus or conditions. In yet another embodiment, the balloon member is a part of a balloon catheter and the system further comprises an oscillating fluid pressure source communicating with the balloon catheter, and a controller configured to cause the oscillating fluid pressure source to deliver controlled pressure oscillations to the balloon member between a maximum pressure equal to the preset maximum inflation pressure and a set minimum pressure at a selected cycle time.

In yet another implementation, the present disclosure is directed to a method of inflating a PTA balloon. The method includes inflating the balloon using an inflation fluid to a selected maximum pressure; delivering controlled pressure oscillations to the balloon through the inflation fluid, the controlled pressure oscillations oscillating at a cycle time of 10 seconds to 0.25 seconds with a pressure reduction between the selected maximum pressure and a set minimum pressure not more than 50% less than the selected maximum pressure.

In still another implementation, the present disclosure is directed to a method of treating vascular disease. The method includes placing a balloon across a lesion in a vessel, the balloon having an outer surface with a nanoparticle matrix disposed thereon, the nanoparticle matrix including drug-carrying nanoparticles; inflating the balloon using an inflation fluid to a selected maximum pressure; delivering controlled pressure oscillations to the balloon through the inflation fluid, the controlled pressure oscillations oscillating between the selected maximum pressure and a set minimum pressure, the oscillations provided at a selected cycle time; and releasing the drug-carrying nanoparticles from the nanoparticle matrix during the delivering, whereby the pressure oscillations facilitate ingress of the drug-carrying nanoparticles into the lesion and surrounding tissue.

In another implementation, the present disclosure is directed to an undulating balloon PTA system. The system includes a balloon catheter including at least one balloon having a preset maximum inflation pressure; a manually actuatable fluid pressure source communicating with the balloon catheter and comprising a syringe body and plunger received in the syringe body; an oscillating fluid pressure source communicating with the balloon catheter; a drive motor powering the oscillating pressure source; and a controller configured to control the drive motor to cause the oscillating pressure source to deliver controlled pressure oscillations to the balloon catheter, the control comprising - delivering fluid pressure oscillations between a maximum pressure equal to the preset maximum inflation pressure and a set minimum pressure not more than 50% less than the maximum pressure; and delivering the fluid pressure oscillations at a selected cycle time in the range of about 10 seconds to about 0.25 seconds.

In still yet another implementation, the present disclosure is directed to an undulating balloon PTA system. The system includes a balloon catheter including a balloon member comprising a double balloon with a first inner balloon inside a second outer balloon, the balloon member having a preset maximum inflation pressure; a manually actuatable fluid pressure source communicating with the first inner balloon and second outer balloon; an oscillating fluid pressure source communicating with second outer balloon; and a controller configured to cause the oscillating fluid pressure source to deliver controlled pressure oscillations to the second outer balloon between a maximum pressure equal to the preset maximum inflation pressure and a set minimum pressure at a selected cycle time.

In a further implementation, the present disclosure is directed to a balloon PTA system. The system includes a multi-segmented balloon catheter. The balloon catheter includes an inflatable balloon member having plural balloon segments arranged along its length; and a catheter body defining a guidewire lumen and plural inflation lumens with one inflation lumen for each the balloon segment, each inflation lumen having at least one inflation port providing fluid communication between a balloon segment and the corresponding inflation lumen, whereby each balloon segment is independently inflatable via separate inflation lumens in the balloon catheter.

In yet another implementation, the present disclosure is directed to an undulating PTA balloon system. The system includes a self-oscillating balloon catheter. The self-oscillating balloon catheter includes a catheter body; a balloon member disposed at a distal end of the catheter body; at least one plunger disposed inside the balloon member; and an actuatable biasing element acting on the at least one plunger configured to cause oscillations of the at least one plunger when actuated.

### BRIEF DESCRIPTION OF THE DRAWINGS

For the purpose of illustrating the disclosure, the drawings show aspects of one or more embodiments of the disclosure. However, it should be understood that the present disclosure is not limited to the precise arrangements and instrumentalities shown in the drawings, wherein:

FIG. 1 is a schematic view of an undulating balloon system.

FIG. 1A is a schematic view of an alternative undulating balloon system configured to control a multi-segmented balloon device.

FIG. 2 is a schematic cross-sectional view of an embodiment of a single balloon device.

FIG. 3 is a schematic cross-sectional view of an embodiment of a double balloon device.

FIG. 3A is a cross section through line A-A of FIG. 3.

FIG. 4 is a schematic cross-sectional view of an embodiment of a multi-segmented balloon device.

FIG. 5 is a schematic cross-sectional view of an embodiment of an internally biased balloon device.

FIG. 6 is a perspective view of an embodiment of an oscillating fluid pressure source in the form of a motorized syringe pump.

FIG. 6A is a perspective view of an alternative embodiment of a motorized syringe pump.

FIG. 6B is a schematic diagram of an alternative embodiment of an oscillating fluid pressure source.

FIG. 7 is a depiction of an embodiment of a user interface for disclosed systems.

FIG. 8 is a flow diagram illustrating an embodiment of a method for control and drug delivery with an undulating balloon.

FIGS. 9A, 9B, 9C, and 9D show different pressure waveforms employed in different treatment algorithms in methods of the present disclosure.

FIGS. 10A and 10B schematically depict one embodiment of a nanoparticle coating with interstitial bonding agent.

FIGS. 11A,11B, 11C, and 11D schematically depict alternative embodiments of a nanoparticle coating with another interstitial bonding configuration.

FIGS. 12A and 12B schematically depict a further embodiment of a nanoparticle coating with a further interstitial bonding configuration.

FIGS. 13A and 13B schematically depict different alternative embodiments of nanoparticle coatings with different interstitial bonding configurations.

FIG. 13C illustrates another alternative embodiment of a balloon device with conductive filaments configurable as sensing means.

FIGS. 14A and 14B schematically depict yet another embodiment of a nanoparticle coating with a different interstitial bonding configuration.

FIG. 15 is a schematic side view of a further balloon embodiment with a heterogenic coating.

### DETAILED DESCRIPTION

Systems and methods for localized drug delivery via drug coated balloons (DCB), in particular using functionalized nanoparticles as a drug delivery medium in combination with an undulating balloon, are disclosed. In various disclosed embodiments, a nanoparticle matrix is adhered to an external substrate-surface, such as the balloon surface, and is activated for release once at the treatment site. Activation for release may be enhanced through the use of an undulating balloon system including methodologies for precise control of timing, waveform and extent of undulations. Certain aspects of the present disclosure may also have applicability in non-undulating drug-coated balloons, plain old balloon angioplasty (POBA), and/or other medical devices placed in the vasculature, such as, for example, stents. Another aspect of the oscillations described herein is to create microchannels in the vessel lumen to enable released nanoparticles to diffuse into the underlying tissue.

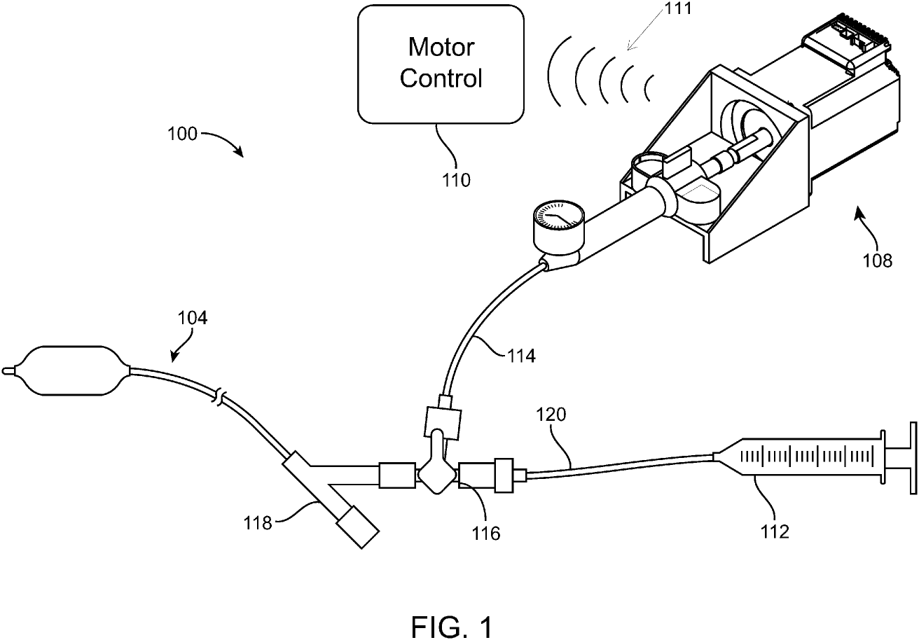

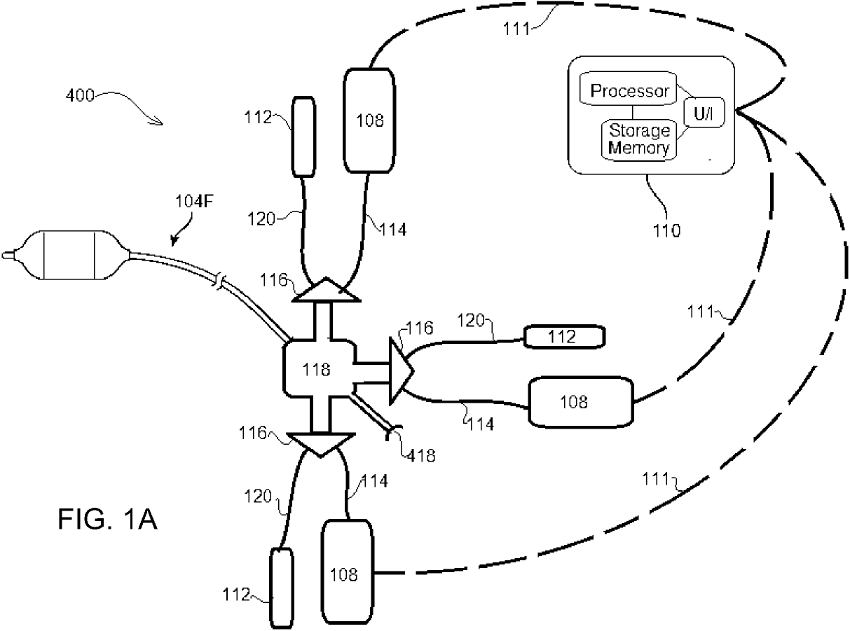

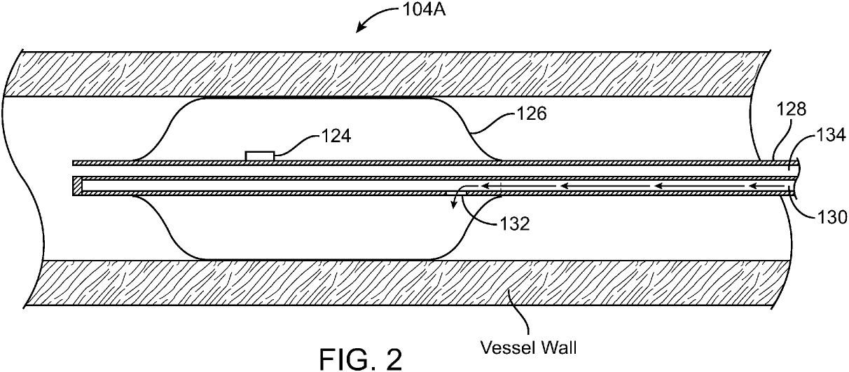

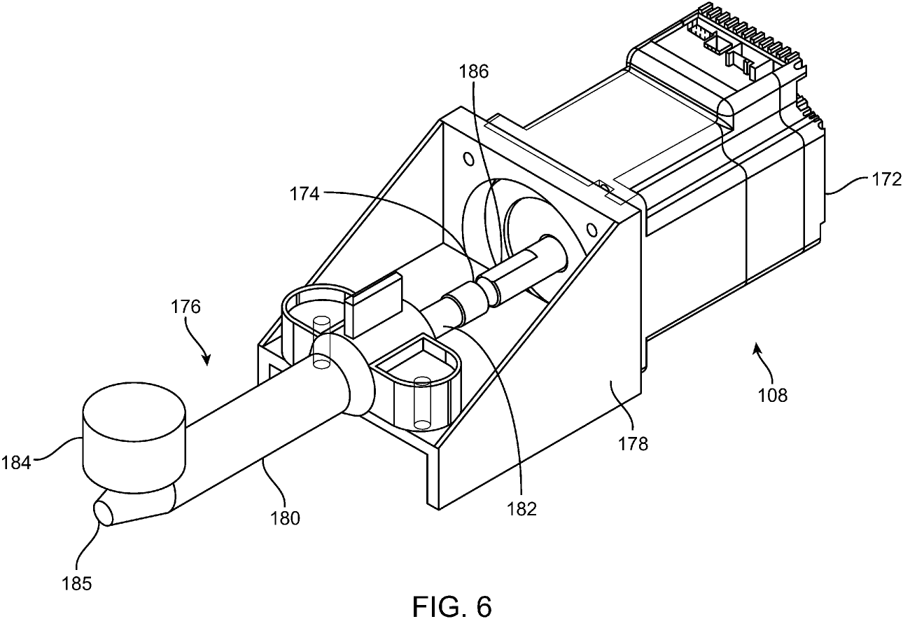

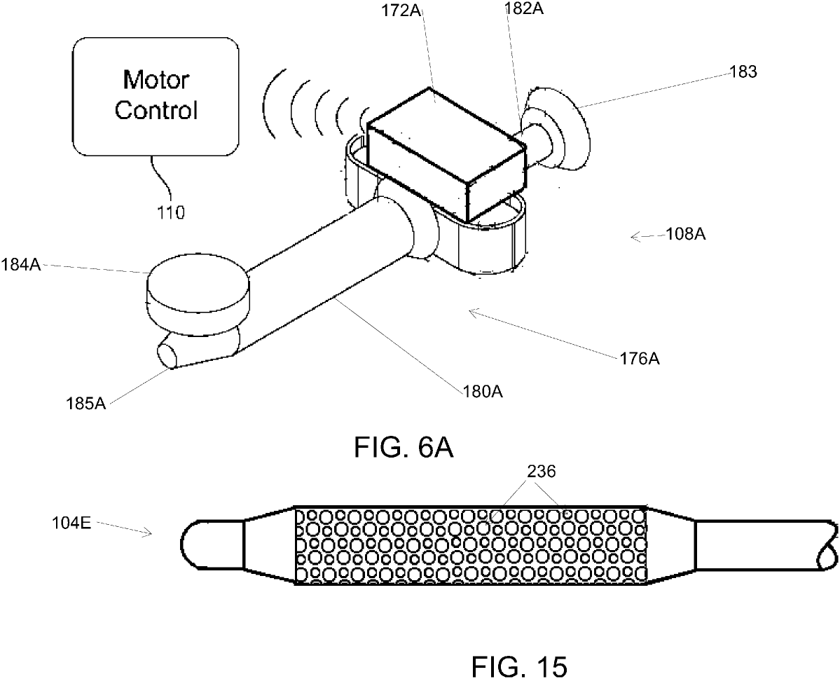

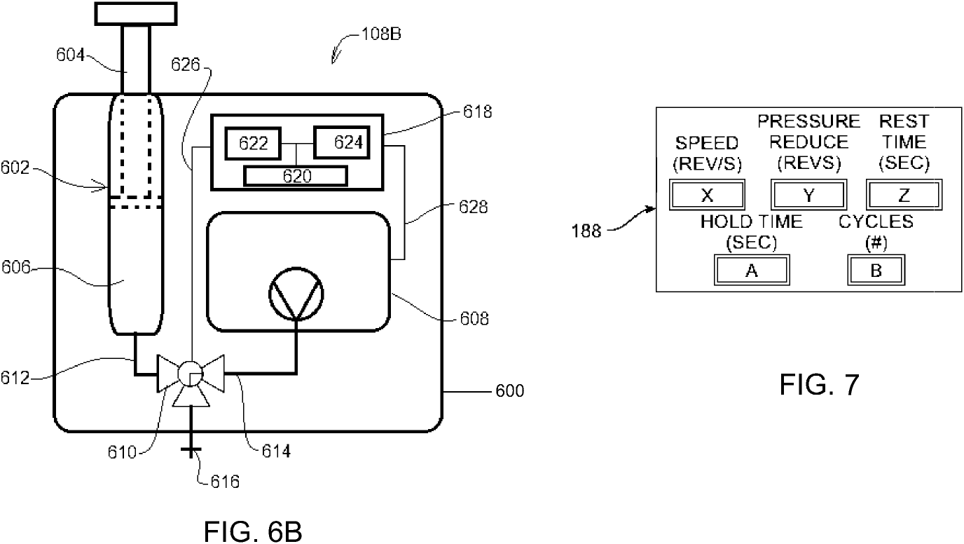

An example of an embodiment of an undulating DCB system is shown in FIG. 1. As shown therein, system 100 includes undulating percutaneous transluminal angioplasty (PTA) balloon 104. Undulating PTA balloon 104 may comprise any of balloon embodiments 104A, 104B, 104C, 104D, or 104E, shown in FIGS. 2, 3, 4, 5, 13C and 15, respectively, or may involve other balloon configurations as may be devised by persons of ordinary skill in the art based on the teachings of the present disclosure. Other components of system 100 include oscillating fluid pressure source 108, controller 110 and syringe 112. Oscillating fluid pressure source 108 communicates with balloon 104 via inflation line 114, three-way stop cock 116 and y-connector 118. Syringe 112, which may in some embodiments provide saline and/or a contrast agent, communicates with balloon 104 via saline/contrast line 120, another branch of three-way stop cock 116 and y-connector 118. In the illustrated embodiment, oscillating fluid pressure source 108 provides controlled pulsations of inflation fluid to create an oscillating pressure profile to induce undulation in the balloon at the treatment site as described further below. More detail with respect to oscillating fluid pressure source 108 is shown in FIG. 6. Alternative sources of controlled periodic pressure oscillations include alternative oscillating fluid pressure source 108A (FIG. 6A) and other periodic pressure sources as may be devised and controlled by persons skilled in the art based on the teachings presented in this disclosure.

In some embodiments, undulating PTA balloon 104 oscillates within a diameter range of about +/-50%, with a preferable diameter oscillation range between a ratio of about 1:1 to 1:2.5 of the initial vessel diameter. In an illustrative example, if the vessel is measured at 6.0 mm, the diameter of the balloon would have a maximum oscillation range between 4.5 mm and 7.5 mm. In smaller vessels, the upper diameter ratio may be about 1: 1.2 or less and in more moderately sized vessels, such as some peripheral arteries, the upper diameter ratio may be about 1:3 to about 1:6. While some degradation or destruction of the plaque may be a beneficial side effect, the oscillations need not be sufficient to break calcium in atherosclerotic plaque. In fact, excessive frequency or amplitude may promote intimal thickening or induce proliferation of smooth muscle cells in the vessel wall tissue, which is to be avoided. Instead, oscillation frequency and amplitude is controlled to more gently introduce microchannels for drug delivering nanoparticles to diffuse through the endothelium and into the underlying vessel wall. Low amplitude pressure cycles comprising a fraction of the maximum inflation pressure are preferred. For example, in a balloon with a 20 atm maximum inflation pressure (burst pressure may be higher), low amplitude pressure cycles would cycle between about maximum pressure of 20 atm and a minimum cycle pressure of not more than a 50% pressure reduction, or about 10 atm minimum pressure. In some embodiments, the maximum pressure reduction should be about 30%, for a minimum cycle pressure of about 14 atm in 20 atm maximum pressure balloon. In other embodiments the maximum pressure reduction should not exceed about 20%, to give a minimum cycle pressure of about 16 atm in the same balloon. Cycle times may exceed times achievable by conventional manual inflation techniques, but should not significantly exceed those levels, and high frequency cycles are to be avoided due to the likelihood of triggering proliferation of smooth muscle cells in the vessel wall at or around the treatment site. Therefore lower frequency pressure cycles are preferred, with typical cycle times, i.e., time between adjacent maximum pressure peaks, typically not below about 0.25 seconds per cycle. In general, the applicable range of cycle times is about 10 seconds down to about 0.25 seconds per cycle. In certain embodiments, it will be desirable to limit the minimum cycle time to be greater than about 0.5 second per cycle. In some embodiments, drug uptake may be increased with greater numbers of cycles, in which case it may be desirable to reduce maximum cycle time to about 1 cycle per second. In such embodiments cycle time may range from 1 to 0.25 seconds per cycle or in others from about 1 to about 0.5 seconds per cycle. With the described relatively lower frequency and pressure amplitude changes, devices disclosed herein are configured to impart micro or nanochannels into the endothelium, while minimizing or eliminating triggering of undesirable cell proliferation (e.g. smooth muscle cells), to allow increased uptake of drug cargo (for example via functionalized nanoparticles as described below) into the underlying tissue while not adding significant injury to the angioplasty procedure.

In another embodiment, fluid displacement is provided by a modified inflator device with a moving piston that changes the pressure automatically for a certain period of time using a mechanized leadscrew and internal pressure sensor. A variety of different balloon types may be used. FIG. 2 shows an embodiment of a simple, single balloon catheter 104A. In this embodiment balloon member 126 is disposed at the distal end of dual lumen catheter body 128. Lumen 130 provides an inflation pathway to balloon member 126 via inflation port 132. Lumen 134 is a guidewire lumen. Catheter body 128 cooperates with a y-connector luer-type fitting as is known in the PTA art. Balloon member 126, and other disclosed balloon embodiments, may be formed of known PTA balloon materials, such as PVC, cross-linked polyethylene, PET or nylon, and may be configured as a compliant or non-compliant balloon. In a further alternative, transducer 124 may be optionally included in any balloon catheter embodiment disclosed herein. Transducer 124 is configured to release energy such as light or ultrasound that is modulated to activate an interstitial bonding layer or nanoparticles-holding matrix of drug carrying nanoparticles on the balloon surface as further described below. When the interstitial bonding layer is activated, it releases the drug carrying nanoparticles from the surface. In general, it is preferred that the energy delivered by transducer 124 be modulated at a level sufficient to activate the targeted bonding layer or nanoparticles, but maintained below a level that would cause an effect on tissue of the vessel wall beyond the balloon and nanoparticle matrix adhered to the balloon substrate. In other embodiments, based on specific clinical objectives, it may be desirable to increase energy levels so as to additionally promote in the surrounding vessel tissue.

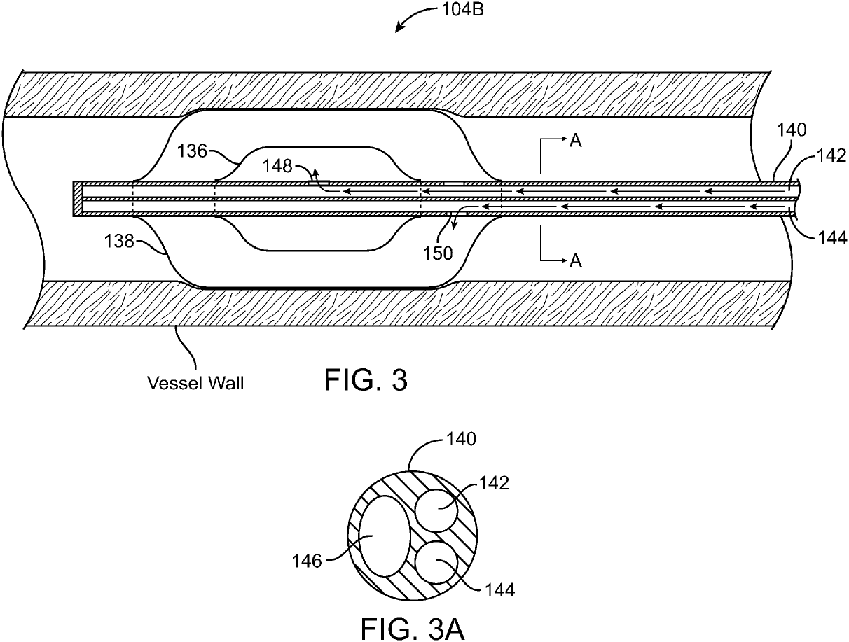

FIG. 3 shows another embodiment of a balloon device for imparting controlled undulations to the vascular wall. As shown therein, dual balloon catheter 104B comprises a balloonin-balloon configuration, whereby inner balloon 136 may be inflated at nominal pressure within outer balloon 138. Triple lumen catheter body 140 provides inner balloon inflation lumen 142, outer balloon inflation lumen 144, and guidewire lumen 146 (FIG. 3A). The space between inner balloon 136 and outer balloon 138 is inflated with a conventional incompressible PTA fluid (contrast media, saline, etc.) or, in some embodiments, based on specific clinical conditions and needs, may be inflated using compressible fluid (e.g. CO₂ or Nitrogen). The fluid between the two balloons pushes the outer balloon to achieve the desired overstretch (e.g. ratio in range of 1:2.5). This configuration may allow for a faster time-constant and response time, because the amount of fluid to be displaced to achieve the nominal outer diameter of outer balloon 138 can be substantially reduced as compared to a single balloon embodiment of the same nominal outer diameter. In one further example, such displacement can be achieved using a proximal end-chamber with a diaphragm. Inner balloon inflation port 148 provides fluid communication between inner balloon 136 and its inflation lumen 142. Similarly, one or more outer balloon inflation ports 150 provide fluid communication between the outer balloon 138 and its inflation lumen 144. The deflection of the vessel wall in contact with outer balloon 138 as shown in FIG. 3 is exaggerated to indicate the overall effect. In another alternative, a selector valve may be provided at the fluid source/proximal end to allow inner balloon inflation lumen 142 to communicate with a constant pressure source, such as indeflator 176 in the manual actuation mode only, while allowing outer balloon inflation lumen 144 to communicate with indeflator 176 in both manual and motor driven modes of an oscillating fluid source such as motorized syringe pump 108A.

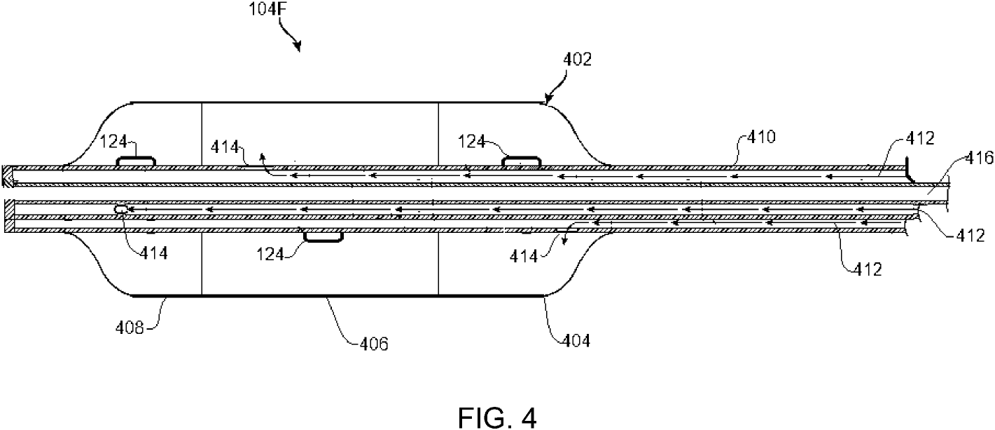

PAD may occur over relatively lengthy sections of arteries, sometimes lengths of 200 mm or more. The characteristics or extent of lesions across lengthy sections of PAD, however, may not be uniform or consistent. Therefore, in the treatment of PAD, it may be desirable to provide lengthy devices, i.e., lengths of 50 mm, 100 mm, or 200 mm or more. Also, due to nonuniformity of lesions in such lengthy sections of disease, it may be desirable to provide a treatment device that offers different levels or characteristics of treatment in different segments of the device. Multi-segmented balloon 104F, as shown in FIG. 4, illustrates an embodiment of such a device. As shown therein, balloon member 402 comprises three separately controllable segments, proximal balloon segment 404, mid-balloon segment 406 and distal balloon segment 408. Inflation of each balloon segment is separately controllable via separate inflation lumens 412 in catheter body 410. Each inflation lumen has an inflation port 414 for each balloon segment. As in typical PTA balloons, guidewire lumen 416 is provided to facilitate placement. In a further alternative, one or more segments may be provided with independently controllable transducers 124 to provide independent modulation and control of nanoparticle matrices in each balloon segment as elsewhere described in the present disclosure.

FIG. 1A illustrates an embodiment of an alternative system 400, configured to control therapy delivery with a multi-segmented balloon such as balloon 104F. It will be appreciated by those skilled in the art that a three segment balloon as shown herein is just an illustrative embodiment, and that any number of segments from two to more than three may be configured based on the teachings of the present disclosure. Multi-segment balloon system 400 includes the same basic components as system 100, just in numbers matched to the number of balloon segment. In this case, however, y-connector 118 includes three branches for balloon inflation plus a guidewire port 418. Controller 110 typically will include at least one processor, a memory and/or storage containing control/therapy algorithm instructions, and details regarding sensed conditions from system sensor. It may also include instructions for control of transducers 124 when present. Controller 110 may communicate with oscillating fluid sources such as motorized syringe pumps via wired or wireless communication links 111.

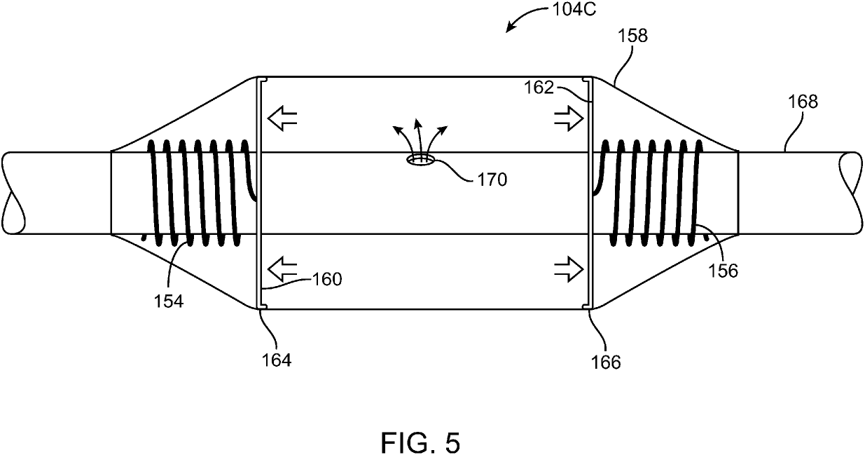

In another alternative embodiment, as shown in FIG. 5, longitudinally biased balloon 104C includes internal biasing elements 154, 156 disposed at opposite ends within balloon member 158. The biasing elements act on plungers 160, 162, respectively, which have seals 164, 166 around their outer periphery configured to at least substantially fluidly seal against balloon member 158 around its inner circumference while allowing longitudinal sliding motion with respect thereto. Catheter body 168 may include at least one inflation lumen and one guidewire lumen (not shown, similar to lumens 130 and 134 in FIG. 2, for example). Inflation port 170 provides inflation fluid to the interior of balloon member 158 via communication with the inflation lumen. In operation, biasing elements 154, 156, together with plungers 160, 162, initially isolate the inflation of balloon member 158 to the longitudinal portion defined between the plungers when the biasing elements are at full extension. Inflation fluid first fills the central portion of the balloon and then the pressure of the inflation fluid acts against the plungers and biasing elements to cause the fully inflated portion of the balloon to grow in the longitudinal direction both distally and proximally.

In another embodiment, the fluid oscillations are created through fluid displacement inside the balloon itself. Such an embodiment may have essentially the same structure as balloon 104C, shown in FIG. 5, with only minor variations. In such an embodiment, fluid displacement inside the balloon may be created through a piston or diaphragm displacement within the fluid in the catheter line, such as plungers 160, 162. Also, rather than acting as passive elements, biasing elements 154, 156 are actively controlled, such as by induction via current applied through control wires (not shown) embedded in the catheter body. Inflation port 170 is used to provide the initial baseline inflation, after which fluid undulation is created by actuating the biasing elements to oscillate the plungers along the linear direction. The balloon may be configured to locate the fluid displacement in a particular segment or as originating from a particular segment. In one example, the fluid displacement occurs proximal-end toward the distal-end of the catheter. In an alternative embodiment, at the distal or proximal-end, the catheter hub has a resonating chamber vibrating a diaphragm or a piston, which provides a substantial displacement. Resonance can be provided by pulsed compressed air or other pulsed fluid. Whether designed with active or passive biasing elements, plungers 160, 162 may be configured as collapsible in segments to facilitate deployment and removal of the device in the patient’s vasculature.

One example of an oscillating fluid pressure source to drive balloon undulation according to the present disclosure is shown in FIG. 6. As illustrated therein, motorized syringe pump 108 comprises drive motor 172, which may be for example a stepper motor, linkage 174, indeflator 176, and mounting bracket 178. Indeflator 176 includes syringe body 180 with internal threads that cause threaded plunger 182 to advance or retreat a precise amount with each rotation of the plunger. Pressure gauge 184, mounted at the distal end of the syringe, measures fluid pressure applied in the system. Tube fitting 185, such as a luer lock fitting, secures inflation line 114 (FIG. 1). Mounting bracket 178 holds indeflator 176 in a fixed position with respect to drive motor 172. Linkage 174 provides a rotating, sliding coupling between drive motor output shaft 186 and threaded plunger 182. Rotation of shaft 186 in either direction thus rotates the threaded plunger while allowing it to freely move in and out of the syringe body.

Another example of an oscillating fluid pressure source is shown in FIG. 6A. In this embodiment motorized syringe pump 108A includes indeflator 176A with integrally mounted drive motor 172A and a manually actuatable threaded plunger 182A. Control knob 183 allows the physician to manually set the initial pressure, whereafter controller 110 controls pressure cycles in accordance with a selected algorithm. In one implementation, drive motor 172A may engage threaded plunger 182A with a ratcheted gear mechanism (not shown) to allow both motorized and manual control of the threaded plunger depth. Other components of motorized syringe pump 108A include syringe body 180A, pressure gauge 184A and tube fitting 185A, which are configured substantially as previously described with respect to motorized syringe pump 108.

Drive motors 172/172A are controlled by controller 110 as shown in FIG. 1. Motor controller 110 may comprise programmable processor-based controls or may be hardware or firmware based with fixed drive profiles. Controller 110 may communicate with drive motor 172 via wired or wireless communication links 111. Table 1 provides illustrative examples of pressure wave form profiles that may be set by controller 110.

TABLE 1

Inflation/Undulation Profiles

| | | | | |
|---|---|---|---|---|
| Wave Form | Pressure Reduction | Rate | Hold Period | Rest Period |
| Square | 10 atm | 1 sec | 20 sec | 30 sec |
| Square | 3 atm | 1 sec | 20 sec | 30 sec |
| Triangle | 10 atm | 5 sec | 1 sec | 25 sec |
| Triangle | 3 atm | 5 sec | 1 sec | 25 sec |
| Sine | 10 atm | 10 sec | 0 sec | 10 sec |
| Sine | 3 atm | 10 sec | 0 sec | 10 sec |

A further alternative embodiment of a periodic fluid pressure source is shown in FIG. 6B. As shown therein, periodic fluid pressure source 108B is provided as an integrated unit. Contained within housing 600 are manually operated syringe pump 602, which comprises threaded plunger 604 received in syringe body 606. Threaded plunger 604 extends out of housing 600 to permit manual adjustment by the physician. Fluid pressure oscillator 608 may comprise any of a variety of approved fluid pump types, such as membrane pumps, elastomeric pumps, or syringe pumps, or it may comprise an oscillating diaphragm. A motor appropriate to the pump type is included in fluid pressure oscillator 608. Both manual syringe pump 602 and fluid pressure oscillator 608 fluidly communicate with three-way valve 610 via fluid lines 612 and 614, respectively. Three-way valve 610 also provides an output port 616 configured with a tube connector such as a luer lock fitting. Delivery of fluid and fluid oscillation is controlled by controller 618, which may comprise processor 620, memory/storage 622 and user interface 624, as may be configured by persons of ordinary skill based on the teachings contained herein. Memory/storage 622 may contain instructions for execution by processor 620 to cause periodic fluid pressure source 108B to deliver fluid in accordance with algorithms disclosed herein. Controller 618 controls the position of three-way valve 610 via communication link 626 and controls fluid pressure oscillator 608 via communication link 628. Manual syringe pump 602 may be provided with sensors (not shown) also communicating with controller 618, such as plunger position indicator or fluid pressure sensor. Alternatively, a fluid pressure gauge communicating with controller 618 may be fitted to output port 616.

In some embodiments, controllers 110, 618 may comprise a user interface that permits user selection of motor drive parameters. FIG. 7 illustrates one embodiment of such a user interface. As shown therein, user interface 188 allows the user to select and set values for motor speed in revolutions per second, pressure reduction for each oscillation in revolutions of threaded plunger 182, rest time between pressure cycles, hold time at maximum pressure in each cycle and number of cycles. Table 2 includes illustrative ranges as for these parameters as may be set by the user in some embodiments.

TABLE 2

Illustrative Control Parameters

| | |
|---|---|
| Parameter | Value Range |
| Motor Speed | X= (varies per device) |
| Pressure reduction | Y= up to about 50% below max pressure |
| Rest time | Z= 0-180,000 ms |
| Hold Time | A= 0 –180,000 ms |
| Cycles | B= 0.1-4.0 Hz |

In specific embodiments, the ranges of rest and hold times may be more narrowly set than the overall ranges shown in Table 2. For example, in some embodiments, rest time may not exceed 1000 ms.

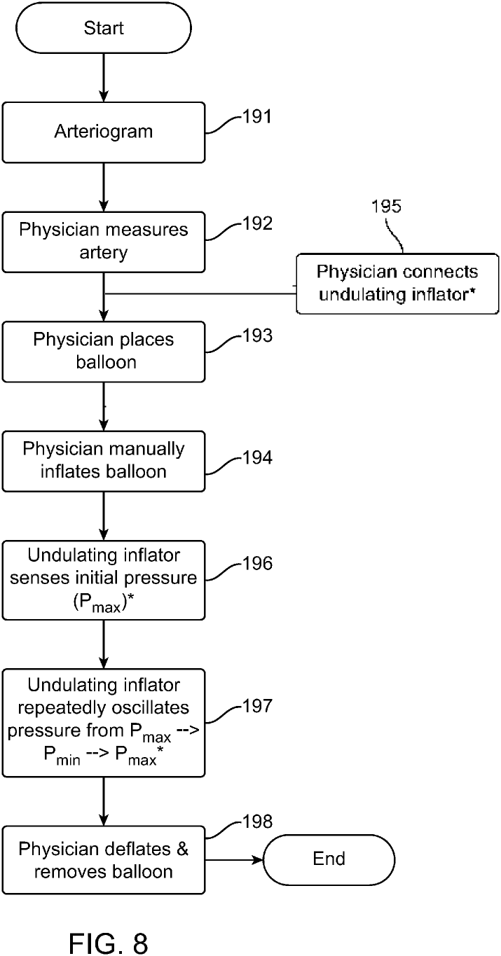

In another aspect of the present disclosure, a method for deployment and treatment with undulating balloon embodiments as disclosed herein includes steps as illustrated in FIG. 8. In general, the physician initiates a procedure following clinical best practices to identify the lesion and navigate all necessary equipment to the deployment site. This may include an arteriogram 191 to identify locations of disease and then using conventional techniques the physician determines the arterial dimensions 192 at the disease sites to be treated so as to select the appropriately sized device(s). After all appropriate patient prep steps consistent with PTA best practices, the physician connects 195 the balloon catheter to an indeflator configured as a periodic pressure source as disclosed herein (e.g. oscillating fluid pressure sources 108 or 108A). the physician navigates 193 the balloon (104) to the treatment site. Typically this will include delivery over a guidewire deployed through the guidewire lumen of one of the disclosed balloon embodiments (e.g., 104A, 104B, 104C, 104D, 104E or 104F).

After spanning the lesion at the treatment site, the balloon is then first inflated to an initial pressure 194 as determined by compliance charts per standard clinical practice based on factors such as vessel size, lesion characteristics and/or balloon size. Such information may optionally be stored in memory or a storage module of controller 110. The initial pressure is typically selected by the physician to correspond to a clinically appropriate maximum balloon diameter as determined by the physician based on measurements made prior to balloon placement. The maximum inflation pressure will not exceed the burst pressure for the balloon. In some embodiments, inflation to the initial pressure may be done manually by the physician using a manual actuator on the indeflator, in others it may be part of the automated control algorithm. In preferred embodiments, the system senses when the initial pressure is reached and sets that pressure as Pₘₐₓ 196. After holding at Pₘₐₓ for a set hold time, the system then controls pressure delivered by the indeflator to oscillate down to either a pre-set reduced value or a physician-determined value, Pₘᵢₙ, before returning to Pₘₐₓ and then oscillating between max and min pressure over selected cycle, hold and rest times 197. Pₘᵢₙ may be determined from the DCB compliance chart to maintain contact between the balloon and the vessel wall to prevent blood flow across the lesion during undulations. Pₘᵢₙ may also be determined angiographically. Pₘᵢₙ may also be determined by the undulating inflator based on pressure or another measurement of balloon-tissue contact such as tissue strain or conductivity. Pₘᵢₙ may also be set to enable blood flow between balloon and vessel wall to aid with rehydration of the nanoparticle coating and thus improve transfer between balloon and vessel wall. Once set cycles are completed, the physician removes the balloon in accordance with standard PTA best practices 198.

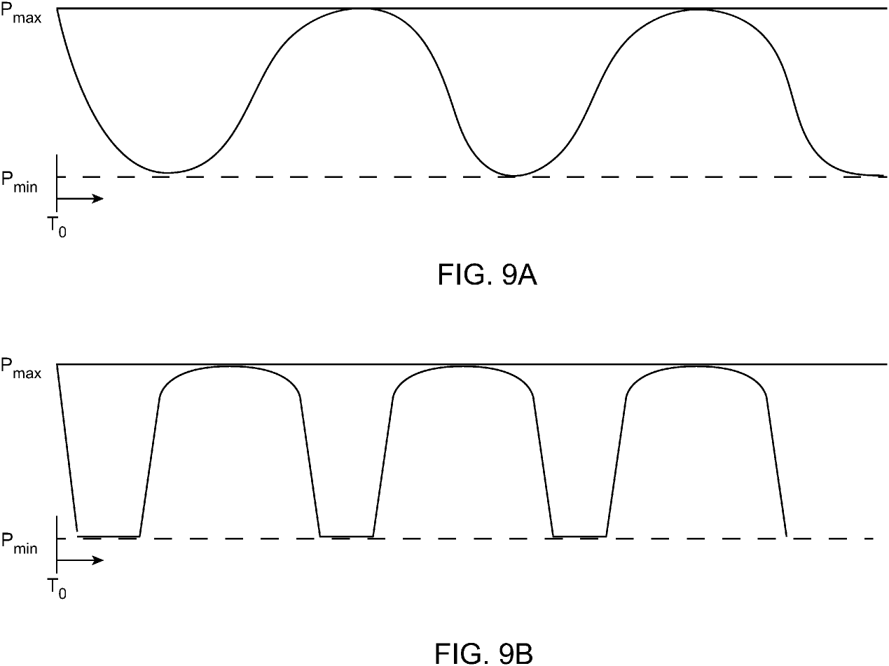

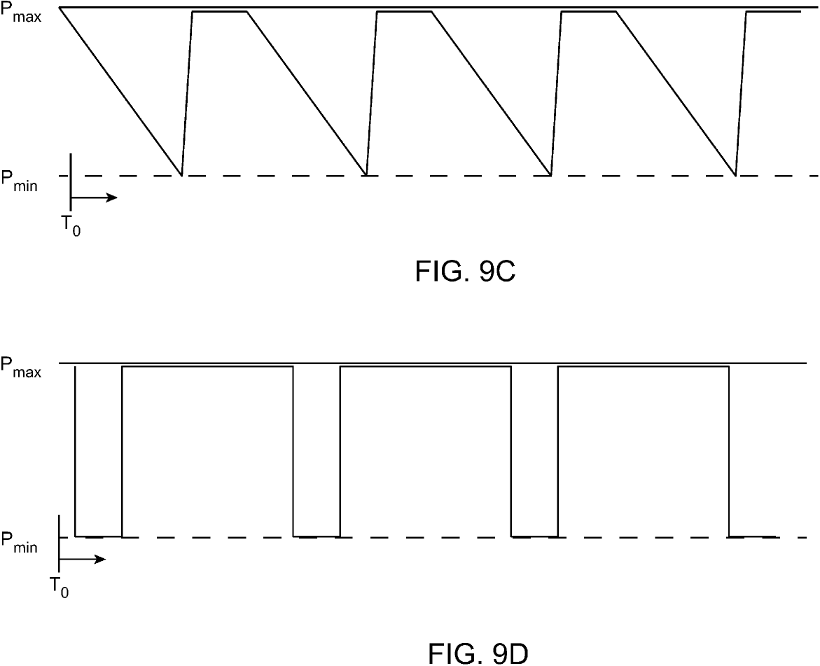

Controller 110 also may be configured with different treatment algorithms employing a variety of different undulation waveforms as shown in FIGS. 9A-D. Different wave forms may be chosen by the physician or pre-programmed for specific types of lesions. Each specific waveform may impart different amounts and extents of microchannels for nanoparticles to traverse the intima depending on the specific properties of the lesion. For example, the extent and location of calcification varies between coronary, peripheral, and below the knee lesions. The saw tooth undulation pattern (FIG. 9C) may work best for below the knee while the sinusoidal wave (FIG. 9A) is sufficient for smaller vessels, such as coronary lesions. The square wave form in FIG. 9D and rounded square waveform in FIG. 9B provide even greater flexibility to design specific treatments. While these waveforms described are a few examples of the possible wave types, sequences, and combinations, the wave shape and pattern are nearly infinite.

The undulation algorithms are preferably configured to optimize disruption of the endothelial layer in different disease profiles. The arterial wall is composed of a plurality of layers, with the endothelial layer being the innermost layer. The undulations are designed to disrupt the endothelial layer (the endothelium and subendothelium). The internal elastic lamina should deform and also form microchannels with more undulations. The combination of these microchannels are believed to increase nanoparticle transport to the smooth muscle cell layer and thus increase tissue concentrations of cargo drug without triggering an intimal thickening response or proliferation of the smooth muscle cells. Specific waveform can be derived to optimally address these factors in each patient/clinical situation by persons skilled in the art based on teachings contained herein.

An important aspect of DCBs is adherence and release of the drug compound on the balloon substrate. Functionalized nanoparticles, for example, as described in the above-incorporated Labhasetwar patent, address the well-known problem of poor uptake of many drugs (e.g. sirolimus), However, such nanoparticles are readily water soluble and therefore require delivery solutions to get past the endothelium to reside in the underlying tissue. The above-disclosed undulating balloons and balloon systems are configured to impart more micro or nanochannels into the endothelium, while minimizing or eliminating triggering of undesirable cell proliferation (e.g. smooth muscle cells), to allow more functionalized nanoparticles into the underlying tissue while not adding significant injury to the angioplasty procedure. Embodiments disclosed herein provide improved coatings and coating techniques for use with disclosed systems and balloons to maximize drug delivery using functionalized nanoparticles as a delivery vehicle. Disclosed devices have characteristics to sustain user-handling while releasing the nanoparticle-carrying matrix or coating when it arrives at the treatment location.

Adhesion of the nanoparticle matrix onto a substrate is dependent on a number of factors, such as particle surface modification and the interface between the substrate and coating. Device manipulation is possible during delivery, but the nanoparticle matrix should remain intact when dry, and then only release within the body (e.g., in vessel or contact with body fluid) due to hydration or other controlled processes as disclosed herein. With respect to hydration, the hydrating characteristics may be selected to achieve such release during an applicable allotted time selected as clinically desirable for a particular patient or treatment. As an illustrative example, DCBs in PAD treatment are typically inflated over about 3 mins, whereas DCBs in coronary disease treatments may be typically inflated for approximately 1 min. In another example, a coronary stent is typically permanently deployed, and has a drug elution time in a range around 90 days. Thus, devices to meet each of these three applications may have multiple different coatings with different hydrating characteristics specific to the application and elution time. The drug elution time designed into a product also may be achieved in whole or in part through the use of an interstitial agent or agents as further described hereinbelow.

In some embodiments, drug elution from the nanoparticle carrier matrix is controlled with the use of one or more interstitial bonding agents between the nanoparticles. Depending on the rate of interstitial bonding agent hydration, a slow or fast release of the drug-carrying nanoparticle coating from the substrate may be achieved. This allows the elution time to be carefully controlled and tailored to the clinical need and, as a result, the nanoparticle coating could be used on a DCB requiring a fast release, or a stent for a slow release by employing the teachings of the present disclosure.

A number of different characteristics of the interstitial bonding agent may be used to tailor the elution time. However, in general, the interstitial bonding agent is selected so as to not degrade the nanoparticles and to preserve the shape, charge, or surface modifier designed into the nanoparticle to create the functionalization of the particles. For example, a water-soluble hydrogel or ductile material bonding agent may be selected with properties such that when dried it allows for bending of the coating without fragmenting the nanoparticle matrix. Examples of materials that may be used as an interstitial bonding agent include PVA, PEG and its co-polymers, PVP, poly-e-caprolactone, chitosan, poly(N-isopropylacrylamide) (NIPAAM), gelatin, poloxamer, alginate, and other similar materials with similar material properties. Such materials may serve as an interstitial excipient for holding the nanoparticle coating together without necessarily interacting with particle transfer to the vessel wall or with cellular uptake of the drug-carrying nanoparticles. However, further modification of the coating material may allow the coating to also serve as an excipient for drug elution, nanoparticle transfer and/or cellular uptake.

Another characteristic of the interstitial bonding agent that may be modulated is its chemical sensitivity to factors such as proteins in the blood, plasma, pH, and cationic and ionic imbalance as means for initiating or promoting degradation. Such a protein-based nanoparticle carrying film may be made out of resilin, elastins including elastin-like polypeptides, silk, collagens, keratins, and bee silks. Protein-based films may employ multiple protein sources with different protein rations to modulate the degradation response initiating factors such as identified above. Multi-layers of hydrophilic and hydrophobic protein materials may be used to create a bond-interface to allow nanoparticles to reside and be released once degraded through pH change, or temperature change or other factors.

Release mechanisms pH sensitivity may employ interstitial bonding agents comprising pH sensitive polymers having a pH critical point designed to obtain a desired change in material behavior - e.g. polyacids with a pH critical point < 7.4 (physiological) would result in a net negative charge on the interstitial bonding agent, such as a non-loaded nanoparticle, causing the polymer to swell and detach from the substrate. Conversely, with polybases if the pKa (i.e. critical point) of the polymer is > 7.4, the polymer will swell upon exposure to blood causing the coating to detach from the substrate. Alternatively, linear block copolymers may be designed to undergo a sol-gel transition such that the properties go from a stiff gel to a soft gel at physiological pH capable of releasing from the substrate. Multi-stimuli polymers may also be used that respond to a combination of both pH and temperature.

Interstitial bonding agents also may be made sensitive to body-temperature by using temperature-responsive polymers within the matrix to change phase when achieving body temperature. A variety of temperature-responsive polymers are available. such as, but not limited to, gelatin, poloxamers (e.g. 407, 127), poly(N-isopropylacrylamide) (NIPAAM), poly(vinylcaprolactame), polyoxazolines (such as poly-2-isopropyl-2-oxazoline), polyvinyl methyl ether, poly[2-dimethylamino)ethyl methacrylate] (pDMAEMA), cellulose-derived polymers (hydroxypropyl myethylcellulose, methyl cellulose, carboxymethylcellulose, ethy (hydroxyethyl) cellulose and the like); xyloglucans, dextrans, poly(g-glutamate), elastin, elastin-like polypeptice/oligopeptide; poly (organophosphazenes), PEG/biodegradable polyester copolymers, PEG-PCL-PEG.

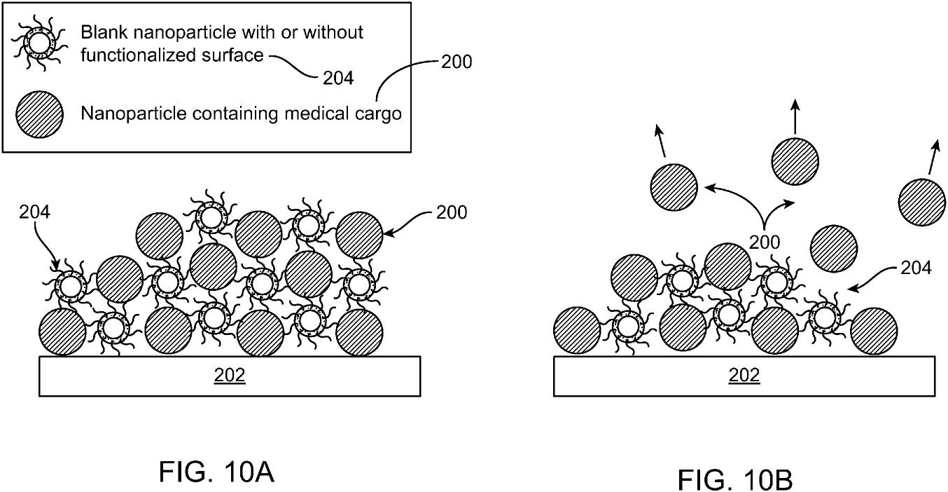

In a further alternative, as illustrated in FIGS. 10A and 10B, medical cargo-carrying nanoparticles 200 are adhered to substrate 202 using an interstitial bonding agent. In one example, interstitial bonding agents may be formed from nanoparticles 204 without a medical cargo (i.e., a blank nanoparticle) with a modified surface that allows interaction and bonding with the nanoparticle-carrying medical cargo. Blank nanoparticle 204 may be configured with a specific degradation time to allow disassociation from nanoparticles 200 carrying medical cargo and releasing the carrying nanoparticles for vessel or tissue absorption as shown in FIG. 10B. Alternative blank nanoparticle embodiments may include micelles or liposome, or organic nanoparticles with an uptake promoting agent (e.g. urea) to provide a release of the nanoparticle and an additional boost in carrying a nanoparticle inside the tissue. As indicated above, substrate 202 may comprise a balloon surface or surface of another medical/vascular device such as a stent.

Blank nanoparticles 204 may be functionalized to provide specific release characteristics using functionalizing elements such as cationic polymers, e.g., poly(ethyleneimine) (PEI), poly-l-(lysine) (PLL), poly-l-arginine, poly[2-dimethylamino)ethyl methacrylate] (pDMAEMA), chitosan, cellulose such as hydroxyethylcellulose, cationic gelatin, dextran, poly(amidoamines), cyclodextrin. Other functionalizing elements for blank nanoparticles 204 may include anionic polymers such as, for example, alginate, carboxymethylcellulose. Functionalized blank nanoparticles 204 may be comprised of dendrimers such as poly(amidoamine) (PAMAM). Dendrimers may be cationic or anionic depending on the surface charge of the nanoparticles. Opposite charged blank nanoparticles 204 will form charge-based interactions with the drug-loaded nanoparticles 200. Degradation of the blank dendrimer will then enable release of the nanoparticle-containing drug. In further alternative embodiments, hydrophobic molecules may be included to encourage hydrophobic interactions between blank nanoparticles 204 and drug loaded nanoparticles 200. Alternatively, hydrophilic molecules encourage hydrophilic interactions between blank nanoparticles 204 and drug loaded nanoparticles 200.

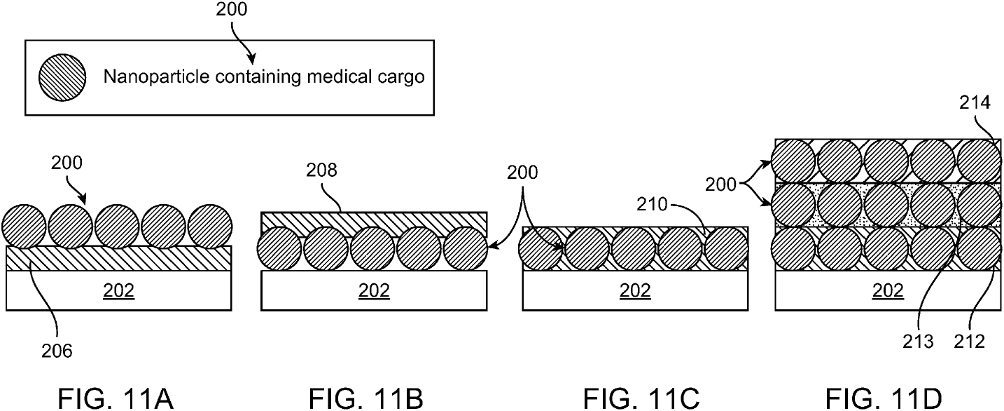

In further alternative embodiments, illustrated in FIGS. 11A-D, the interstitial bonding agent may be formed of or employ a nanofiber, either polymeric or protein-based, applied to a matrix of nanoparticles 200 either as an initial matrix 206 in which nanoparticles are either sprayed or the medical device dipped, or applied as an on-top coating 208 on substrate 202, such as a balloon. In another alternative, interstitial bonding agents may be delivered in mixture 210 with nanoparticles 200 or applied as topcoat 208 during the coating process and after the application of one or more layers of nanoparticles 200. Using multiple coating layers with different characteristics, such as layers 212, 213, 214, each layer in a matrix with drug-carrying nanoparticles 200, a wide range of release times and/or multiple drug releases may be achieved as different layers are designed to release the nanoparticles at different rates, for example, in a nested doll-like configuration. Nanofibers may also be stimuli (e.g. temperature or pH) responsive. Nanofiber can be cationic or anionic depending on charge of drug-loaded nanoparticles to add another level of retention. In another example, layers of different interstitial bonding agents or nanoparticles with the same or different drugs applied one layer at a time, may be used to provide controlled release of the drug. In such an embodiment, the layers may be configured so as to dissolve at the same or different rate depending on the clinical need. Embodiments of this type, for example as shown in FIG. 11D, provide form of a nested doll-like mechanism as the layers are dissolved with different kinetics.

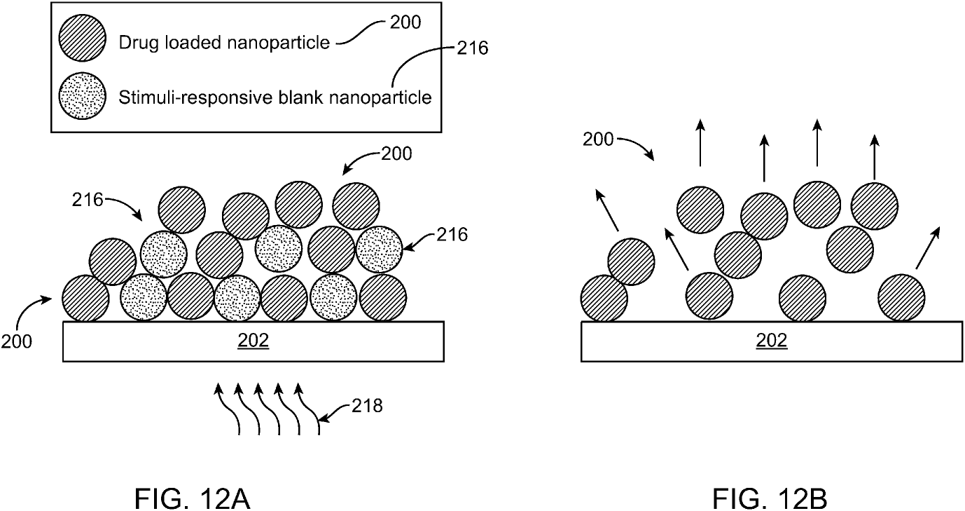

In other alternative embodiments, as illustrated in FIGS. 12A and 12B, the interstitial bonding agent may be of single or several compositions depending on the application needs. For example, the interstitial bonding agent or interstitial bonding gel, or interstitial bonding nanoparticle 216 (collectively, bonding agent generally), can be configured with a sensitivity to a specific triggering mechanism 218 such as, temperature, pH, light or sound sensitivity. For example, light applied either from inside the medical device (inside the balloon, as with transducer 124 (FIG. 2)) or from the outside, may be used to trigger a controlled release of nanoparticles. Light types, such as IR, UV, or regular white illumination, may be employed. If sound sensitive, for example, ultrasound waves may be applied to trigger release by degrading the bonding agent. Ultrasound has an advantage in being well-understood for medical applications and many types of transducers exist that could apply ultrasound energy from inside of the medical device itself (e.g., within the DCB) using internal ultrasound resonators, such as small piezo chips, or from outside using an external source targeting the location of the balloon. In one alternative, ultrasonic sensitive materials are embedded in an angioplasty balloon and activated during the balloon inflation, for example by an internal resonator. In ultrasound embodiments, it may be desirable to limit the ultrasound frequencies to levels less likely to promote undesirable tissue interactions. Thus in some embodiments, as an ultrasound transducer, transducer 124 may be configured to produce ultrasound energy at frequencies under 1 MHz, and in other embodiments, at frequencies under 100 KHz. Similarly, stent deployment with a PTA balloon equipped with ultrasonic resonators may provide such ultrasound for the purpose of breaking down the interstitial bonding agent and release the nanoparticles. The vibration from the ultrasound or high-amplitude vibration with a low frequency in such embodiments are used to either break the interstitial bonding agent or mechanical structure of the nanoparticle matrix.

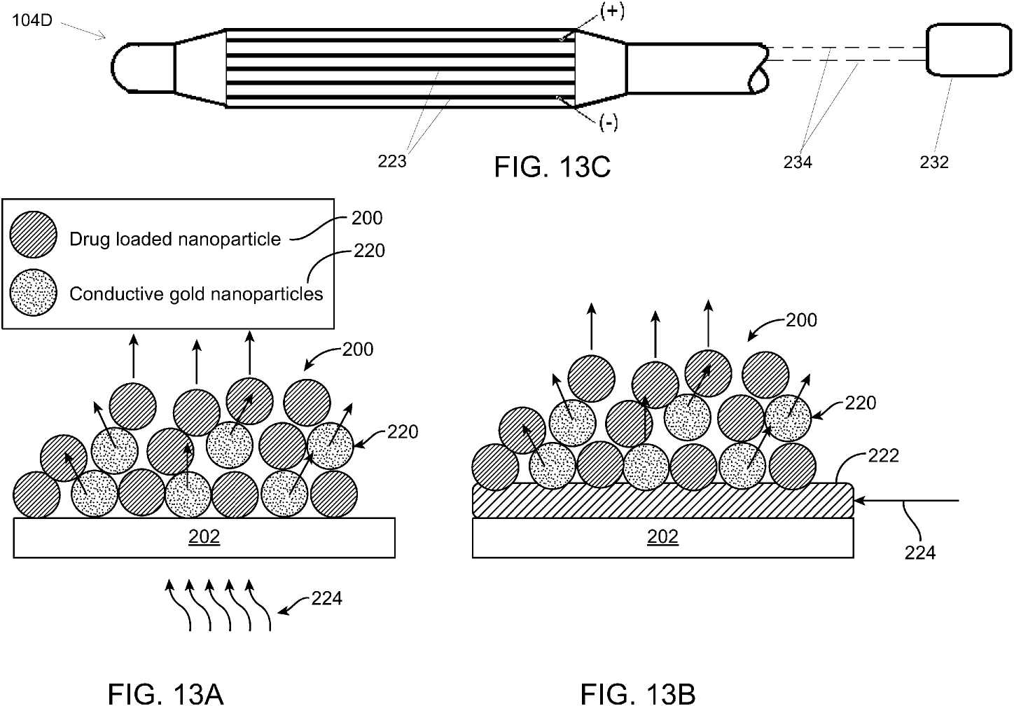

In other embodiments, as illustrated in FIGS. 13A and 13B, electrical sensitivity is used as a triggering means for releasing nanoparticles 200 or degrading the bonding agent. For example, in one embodiment, conductive nanogold particles 220 are interstitially embedded among nanoparticles 200 with a therapeutic cargo. Further, substrate 202 may have a conductive layer 222 applied thereto, such as conductive ink or conductive filaments, through which a current 224 could be driven to deliver a release triggering electrical stimulus. As a further option, the polarity could be reversed creating a repulsion of the nanoparticles attached to nanogold particles 220. The electrical stimulus thus generates the structural degradation of the nanoparticle matrix to release its cargo.

In yet another embodiment, an example of which is illustrated in FIG. 13C, conductive layer 222 may be formed as a plurality of conductive filaments 223 extending along the length of balloon 104D. In one embodiment, conductive filaments may be applied by ink-jet deposition using a conductive ink or ink-like material. With multiple conductive filaments 223 on the balloon, the resistance or impedance between two or multiple filaments can be measured to determine the degree of hydration associated with the coating. In one embodiment, filaments 223 may communicate with power supply/controller 232 through conductors 234 embedded in the catheter body wall. Power supply/controller 232 may be configured as a standalone control device including a user interface and indicator of sensed impedance, or may be integrated with a system controller such as controller 110 (FIG. 1). Conductive filaments 223, with or without the conductive nanoparticle, provide a sensor to help determine the length of time necessary to place the balloon in the artery inflated so that the entire cargo is delivered. With this particular method, the physician may know the coating hydration quality or status when the device arrives at the lesion site and how long to place the device. Also, one can look at the indicator to determine if the washout was too severe and device (e.g., DCB) is no longer viable to be used in the procedure, and thus whether it should be exchanged for another device.

Using a sensor such as formed by conductive filaments 223, the physician also may obtain information via controller 232 indicating when to deflate or remove the device because the nanoparticle matrix has fully degraded. Therefore, with such a sensor underlying the nanoparticle matrices as described herein, and measurement of the impedance change, one can determine when the coating has completely eluted out of a device such as a DCB. Such information, previously unavailable with existing devices, will help adjust the inflation time to either shorten it or extend it as permissible in order to optimize treatment delivery. Currently, DCB inflation time is typically fixed, without real-time feedback on possible effectiveness of drug delivery. For instance, instead of having a fixed time of 3 min during the delivery of the DCB balloon, such time could either increase or decrease depending on the in vivo elution time and the medical decision to do so.

Nanoparticle hydration is another parameter that can be modulated to beneficial effect in embodiments of the disclosed devices. The rate of nanoparticle matrix hydration is a factor in releasing the nanoparticle from the substrate during the desired application time. If duration of an application is of a short time, the nanoparticle matrix should have that brief time to degrade and elute the nanoparticle. At a basic level, the nanoparticle matrix is applied against a vessel wall and drawing water from the environment, which causes the coating to re-hydrate and degrade. Various factors may be employed to modulate the rate of hydration as the mechanism of degradation and thus application times may be manipulated and enhanced. For example, a salt-based polymer embedded with the nanoparticle matrix will augment the ability to attract water faster into coating. Also, controlling blood flow over the substrate surface between pressure cycles can contribute to hydration.

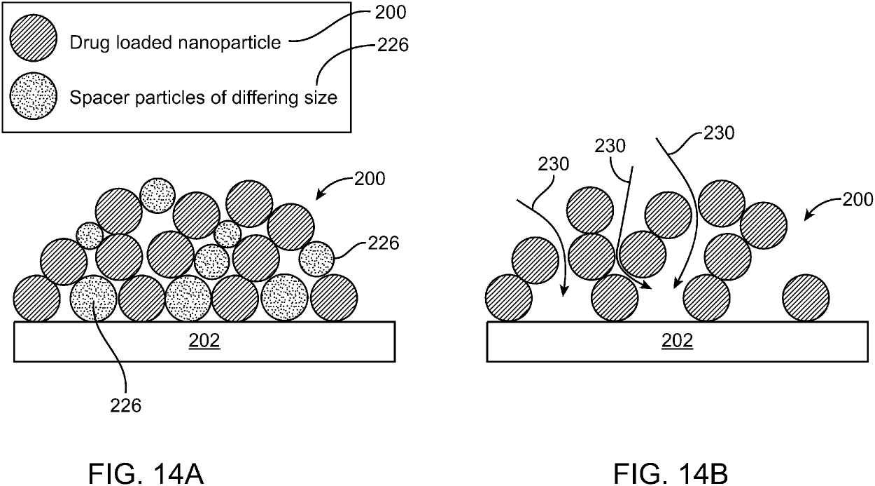

In another alternative, as illustrated in FIGS. 14A and 14B, a nanoparticle matrix can be formed from nanoparticles with different sizes and functions. For example, first nanoparticles 200 with drug loading and a first size are combined with nanoparticles or microparticles 226 of a single different size or varying sizes acting as spacers within the matrix. These spacer-type particles 226 can be removed from the nanoparticle matrix during the manufacturing process through the use of an external agent (e.g., heat and/or vibration to selectively break the structure of particle spacers 226, or vacuum to explode embedded spacer particles), so that the end result is a lattice-shaped matrix with nano or micro-sized channels 230. Such channels 230 provide a capillary effect to attract water inside the matrix. Lattice structures also may be formed using spacer nanoparticles or microparticles with a salt-based cargo. When removed as described above, the salt crystal remains in the lattice with the purpose of attracting water for a manipulated hydration of the coating.

Devices disclosed herein are not limited to delivery of individual drugs. Disclosed devices may employ nanoparticles carrying multiple therapeutic or diagnostic (e.g. cellular tagging) cargo. The conjugation of drug and biodegradable nanoparticles could be done through standard nanoparticle formulation and fabrication as it is known in the field. Employing known techniques as modified by the teachings of the present disclosure, various embodiments may deliver cargos such as, but not limited to anti-inflammation, anti-arrhythmic, anti-proliferative, anti-restenosis, Botox®, cortisone, cytotoxic drugs, and cytostatic drugs. For example, the desired cargo may be mixed with a biodegradable material such as a PLLA/PLGA mixture or other biodegradable material known in the art. The cargo type in such embodiments may be multi-drug in one nanoparticle or a slurry of multiple nanoparticles with a single drug each.

A further aspect of the present disclosure are coating processes for creating nanoparticle layers as described above. In one embodiment, the coating process employs an aqueous solution and uses a lyophilized nanoparticle recombined into a water-based interstitial bonding agent (as described above) or just an interstitial non-bonding agent (water) to form a slurry. The nanoparticles may include a surface modifier to help connect with the substrate’s surface, such surface modification is substrate-dependent and designed to interact with one surface at a time. The slurry is applied to a device surface by deposition, for example using an ink-jet type of deposition, where it is sputtered on to the device surface (e.g., balloon) one layer at a time. In one embodiment, illustrated in FIG. 15, the sputtering is a high frequency dot deposition wherein lines are created on balloon 104E substrate’s surface with different sized or shaped dot depositions 236 such that the lines are heterogenic in nature to create a heterogenic coating. The heterogenic coating creates cavities in the nanoparticle matrix to promote coating hydration and flow of water molecules within the coating. The slurry does not necessarily require a solvent conjugate, but a solvent could be added to accelerate the drying of the sputtered lines.

The slurry with a water-based interstitial bonding or non-bonding agent when deposed using inkjet or inkdot technology may not require an additional drying mechanism (e.g. air flow or heat). In one process embodiment, the coating machine and the catheters are placed in an extremely low humidity environment to extract the water from the coating. Alternatively, the product is placed in a vacuum chamber to extract water out of the coating. Further drying processes such as lyophilization may be employed, i.e. subjecting the coating to a freezing temperature environment or freezing fluid inside the balloon to drive off moisture.

In a further alternative embodiment, during the coating process, the balloons or substrate are purposely undersized. As an illustrative example, for instance, a 6 mm nominal balloon is coated at 4 mm diameter instead of at its nominal diameter. Thus when the device is applied in the clinical setting, the coating is overstretched further from the original coating diameter, providing additional mechanical degradation of the surface layers that allows for water absorption. Alternatively, the balloon may be oversized during coating, e.g. the 6 mm balloon inflated to 8 mm, to create small gaps between lines/dots of the coating to facilitate/channel water ingress.

In another embodiment, the deposition of the coating lines on the substrate is performed with an ink-jet deposition using either an ultrasound-nozzle or a non-ultrasound nozzle with just a spray pattern. With the use of either nozzles, the nanoparticles are designed to sustain the shear stresses during the coating process. For instance, with the nanoparticles are fabricated a solid nanosphere, the surface-modifiers are also designed to sustain high shear stresses. The deposited lines are differentiated such that when the substrate is rotated, there is no effect of gravity in drooping the material from the balloon. This is achieved through a combination of nanoparticle design, interstitial (bonding or non-bonding) agent, viscosity manipulation, and temperature. In a further alternative, using inkjet line deposition, after applying the coating lines the balloon is then frozen to enable use of lower viscosity NP solutions. Thereafter the coating can either slowly thaw to dry or lyophilize, which may impart microchannels for water to invade the coating and enhance release/transfer.

During the coating process, single or multiple sprays from depositing nozzles could be used. The spray nozzles may operate to sputter continuously or intermittently. Using multiple spray nozzles, the content delivered by the nozzles could be the same or different depending on the spraying need. For instance, one nozzle could have a nanoparticle with drug cargo, and the other nozzle could have a topcoat for re-hydration manipulations. Also, a nozzle could have a nanoparticle with Drug A and the other nozzle(s) with a nanoparticle with Drug B or C, etc., netting a balloon with multiple drugs, for instance depositing both Paclitaxel and Sirolimus on the same balloon at different ratios. In one embodiment, a low quantity of paclitaxel and a large quantity of sirolimus. A combination of multiple nozzles also may be employed, for example, one spraying continuously and the other intermittently to create channels for water ingress.

In alternative embodiments, a robotic coating machine may be used with the ability to coat the substrate selectively leaving some areas without any coating. In one example, in a PTA balloon, the cones are not part of the therapy and therefore could be excluded from the coating process. In another alternative, the non-therapeutic area is coated with a hydration-promoting agent, such as a hydrophilic coating. In a further alternative, the topcoat is a degradable hydrophilic coating applied either after folding or before folding the PTA balloon. In yet another alternative embodiment, a dip coating process is used to coat devices such as DCBs, wherein a multiple dip in a same solution or multiple dip in different solutions of distinct actions can be employed.

The foregoing has been a detailed description of illustrative embodiments of the disclosure. It is noted that in the present specification and claims appended hereto, conjunctive language such as is used in the phrases “at least one of X, Y and Z” and “one or more of X, Y, and Z,” unless specifically stated or indicated otherwise, shall be taken to mean that each item in the conjunctive list can be present in any number exclusive of every other item in the list or in any number in combination with any or all other item(s) in the conjunctive list, each of which may also be present in any number. Applying this general rule, the conjunctive phrases in the foregoing examples in which the conjunctive list consists of X, Y, and Z shall each encompass: one or more of X; one or more of Y; one or more of Z; one or more of X and one or more of Y; one or more of Y and one or more of Z; one or more of X and one or more of Z; and one or more of X, one or more of Y and one or more of Z.

Various modifications and additions can be made without departing from the spirit and scope of this disclosure. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present disclosure. Additionally, although particular methods herein may be illustrated and/or described as being performed in a specific order, the ordering is highly variable within ordinary skill to achieve aspects of the present disclosure. Accordingly, this description is meant to be taken only by way of example, and not to otherwise limit the scope of this disclosure.

Exemplary embodiments have been disclosed above and illustrated in the accompanying drawings. It will be understood by those skilled in the art that various changes, omissions and additions may be made to that which is specifically disclosed herein without departing from the spirit and scope of the present disclosure.

1. An undulating balloon PTA system, comprising:
a balloon catheter comprising an inflatable balloon member having a preset maximum inflation pressure;
an oscillating fluid pressure source communicating with the balloon catheter;
a controller configured to cause the oscillating fluid pressure source to deliver controlled pressure oscillations to the balloon catheter between a maximum pressure equal to the preset maximum inflation pressure and a set minimum pressure at a selected cycle time; and
a drug carrying nanoparticle matrix disposed on an outer surface of the balloon member.

2. The undulating balloon PTA system of claim 1, wherein the set minimum pressure is not more than 50% less than the maximum pressure.

3. The undulating balloon PTA system of claim 2, wherein the controller is further configured to deliver the pressure oscillations at a cycle time of 10 seconds to about 0.25 seconds.

4. (canceled)

5. (canceled)

6. (canceled)

7. (canceled)

8. The undulating balloon PTA system of claim 1, wherein the oscillating fluid pressure source comprises a drive motor operatively controlled by the controller.

9. The undulating balloon PTA system of claim 8, wherein the oscillating fluid pressure source further comprises a syringe body with a plunger driven by the drive motor.

10. (canceled)

11. The undulating balloon PTA system of claim 1, wherein:
said balloon catheter includes a balloon member comprising a double balloon with a first inner balloon and a second outer balloon;
said oscillating fluid pressure source communicates with the outer balloon through a first inflation lumen in the balloon catheter; and
a non-oscillating pressure source communicates with the inner balloon through a second inflation lumen in the balloon catheter.

12. The undulating balloon PTA system claim 1, wherein:
said balloon catheter includes a segmented balloon member comprising plural balloon segments, being independently inflatable via separate inflation lumens in the balloon catheter; and
at least one said oscillating fluid pressure source communicates with said balloon segments through said inflation lumens.

13. The undulating balloon PTA system of claim 12, wherein:
the system further comprises plural said oscillating fluid pressure sources; and
each balloon segment inflation lumen communicates with a different said oscillating fluid pressure source, whereby each balloon segment is provided with separately controllable fluid pressure oscillations.

14. (canceled)

15. The undulating balloon PTA system of claim 1, wherein the drug-carrying nanoparticle matrix contains microchannels, whereby blood may circulate in the microchannels to increase hydration of the nanoparticle matrix.

16. The undulating balloon PTA system of claim 1, wherein:
the nanoparticle matrix comprises drug-carrying nanoparticles and interstitial bonding agent; and
the interstitial bonding agent is configured to release the drug-carrying nanoparticles in response to predetermined stimulus or conditions.

17. The undulating balloon PTA system of claim 16, wherein the predetermined condition is a predetermined time in contact with blood within a vessel to be treated.

18. The undulating balloon PTA system of claim 16, wherein the stimulus is temperature or pH.

19. The undulating balloon PTA system of claim 16, wherein:
the stimulus is thermal energy, sonic energy or light energy; and
the balloon catheter further comprises a transducer disposed within the balloon member configured to generate the corresponding stimulus energy.

20. The undulating balloon PTA system of claim 16, wherein the stimulus is sonic energy and the system further comprises an external ultrasound transducer configured to deliver sonic energy at a frequency under 1 MHz.

21. The undulating balloon PTA system of claim 16, wherein;
the interstitial bonding agent comprises conductive particles; and
the balloon member includes at least one conductive element, whereby electrical current delivered through the at least one conductive element releases the drug-carrying nanoparticles.

22. The undulating balloon PTA system of claim 21, wherein the at least one conductive element comprises a plurality of conductive filaments on an outer surface of the balloon member.

23. (canceled)

24. (canceled)

25. (canceled)

26. (canceled)

27. (canceled)

28. A method of treating vascular disease, comprising:
placing a balloon across a lesion in a vessel, the balloon having an outer surface with a nanoparticle matrix disposed thereon, the nanoparticle matrix including drug-carrying nanoparticles;
inflating the balloon using an inflation fluid to a selected maximum pressure;
delivering controlled pressure oscillations to the balloon through the inflation fluid, the controlled pressure oscillations oscillating between the selected maximum pressure and a set minimum pressure, said oscillations provided at a selected cycle time; and
releasing the drug-carrying nanoparticles from the nanoparticle matrix during said delivering, whereby said pressure oscillations facilitate ingress of the drug-carrying nanoparticles into the lesion and surrounding tissue.

29. The method of treatment of claim 28, wherein:
the drug-carrying nanoparticle matrix contains microchannels; and
said releasing comprises allowing blood to circulate in the microchannels to increase hydration of the nanoparticle matrix.

30. The method of treatment of claim 28, wherein the nanoparticle matrix comprises an interstitial bonding agent configured to release the drug-carrying nanoparticles in response to predetermined stimulus or condition.

31. The method of treatment of claim 30, wherein the predetermined condition is a predetermined time in contact with blood within a vessel to be treated and said releasing comprises maintaining the balloon across the lesion for a time at or exceeding the predetermined time.

32. The method of treatment of claim 30, wherein the stimulus is temperature or pH.

33. The method of treatment of claim 30, wherein the stimulus is thermal energy and said releasing comprises directing heat energy at the nanoparticle matrix.

34. The method of treatment of claim 30, wherein the stimulus is sonic energy and said releasing comprises directing sonic energy at the nanoparticle matrix.

35. The method of treatment of claim 30, wherein the stimulus is light energy and said releasing comprises directing light energy at the nanoparticle matrix.

36. The method of treatment of any of claims 30, wherein said predetermined stimulus comprises directing an energy source at the nanoparticle matrix from a transducer disposed within the balloon member.

37. The method of treatment of claim 36, wherein said directing of the energy -source comprises directing said energy at a level selected to disrupt the interstitial bonding agent while minimizing or avoiding triggering proliferation of smooth muscles cells in the vessel wall or thickening of the intimal layer.

38. The method of treatment of claim 30, wherein the interstitial bonding agent comprises a material sensitive to temperature and said releasing comprises introducing the balloon member into the vessel causing a change in property of the interstitial bonding agent, whereby drug-carrying nanoparticles are released from the balloon member surface.

39. The method of treatment of claim 30, wherein the interstitial bonding agent comprises a material sensitive to pH and said releasing comprises introducing the balloon member into the vessel causing a change in property of the interstitial bonding agent, whereby drug-carrying nanoparticles are released from the balloon member surface.

40. The method of treatment of claim 30, wherein the interstitial bonding agent comprises conductive particles and said releasing comprises delivering a current to the balloon member surface to free the drug-carrying nanoparticles from the nanoparticle matrix.

41. (canceled)

42. An undulating balloon PTA system, comprising:
a balloon catheter including at least one balloon having a preset maximum inflation pressure;
a manually actuatable fluid pressure source communicating with the balloon catheter and comprising a syringe body and plunger received in the syringe body;
an oscillating fluid pressure source communicating with the balloon catheter, wherein manually actuatable fluid pressure source and the oscillating fluid pressure source comprise a common syringe body and plunger and actuation of the plunger is switchable between manual actuation and motor driven actuation;
a drive motor powering the oscillating pressure source; and
a controller configured to control the drive motor to cause the oscillating pressure source to deliver controlled pressure oscillations to the balloon catheter, said control comprising -
delivering fluid pressure oscillations between a maximum pressure equal to the preset maximum inflation pressure and a set minimum pressure not more than 50% less than the maximum pressure;
delivering the fluid pressure oscillations at a selected cycle time in the range of about 10 seconds to about 0.25 seconds; and
a drug-carrying nanoparticle matrix disposed on an outer surface of the balloon, the nanoparticle matrix comprising drug-carrying nanoparticles and interstitial bonding agent, wherein the interstitial bonding agent is configured to release the drug-carrying nanoparticles in response to predetermined timulus or conditions, wherein the drug-carrying nanoparticle matrix contains microchannels, whereby blood may circulate in the microchannels to the increase hydration of the nanoparticle matrix.

43-67. (canceled)
